(19) United States
(12) Patent Application Publication
(10) Pub. No.: US 2023/0363932 A1
(43) Pub. Date: Nov. 16, 2023

(54) DISTAL LIMB PERFUSION CANNULA STENT & STENT CANNULA COVER

(71) Applicant: ABIOMED, Inc., Danvers, MA (US)

(72) Inventors: Christopher Korkuch, Danvers, MA (US); Glen Fantuzzi, Danvers, MA (US); Charles DeLorenzo, Danvers, MA (US); Emilia Jahangir, Danvers, MA (US); Matthew D'Agostino, Danvers, MA (US); Robert Swierczek, Danvers, MA (US); Alex McCall, Danvers, MA (US)

(73) Assignee: ABIOMED, Inc., Danvers, MA (US)

(21) Appl. No.: 18/195,535

(22) Filed: May 10, 2023

Related U.S. Application Data
(60) Provisional application No. 63/340,352, filed on May 10, 2022.
(60) Provisional application No. 63/444,529, filed on Feb. 9, 2023.

(51) Int. Cl. A61F 2/954; A61F 2/958

(52) U.S. Cl. CPC A61F 2/954; A61F 2/958

(57) ABSTRACT

A device and method are provided for minimizing and/or preventing limb ischemia, such as when a medical device inserted into a patient's vasculature occludes the vessel. The device may include a tubular member and a first anchor configured to hold a position of the tubular member relative to a vessel. The first anchor is configured to appose an internal wall of the vessel. A second anchor may cooperate with the first anchor to hold the position of the tubular member relative to the vessel.

### CROSS-REFERENCE TO RELATED APPLICATIONS

The present application claims priority to U.S. Provisional Application Nos. 63/340,352, filed May 10, 2023, and 63/444,529, filed Feb. 9, 2023, the contents of which are each incorporated by reference herein in its entirety.

### TECHNICAL FIELD

The present disclosure relates to methods, devices, and systems used in the prevention of limb ischemia, such as that resulting from use of a mechanical circulatory support device.

### BACKGROUND

Limb ischemia is a rapid and sudden decrease in limb perfusion often threatening limb viability. It may occur as a result of blockage of blood due to an indwelling sheath and/or catheter, local occlusion (e.g., atherosclerotic narrowing), and/or continuous occlusion resulting from small vessels and/or large sheaths. It may also occur as a result of a closure issue.

Limb ischemia is associated with mortality, and some literature indicates the rate of limb ischemia may be high. As such, in addition to negatively impacting the patient, it may also negatively impact ongoing clinical trials.

### BRIEF SUMMARY

In various aspects, a device may be provided. The device may include a tubular member configured to extend at least partially into a vessel of a patient. The device may include a first anchor coupled to the tubular member and configured to hold a position of the tubular member relative to the vessel. The first anchor may be configured to appose an inner wall of the vessel.

The first anchor may include a delivery configuration and a deployed configuration. In the deployed configuration, the first anchor may have a diameter greater than an opening of the vessel through which the tubular member extends into the vessel. The first anchor may include a plurality of fingers. The first anchor may be attached at or near a distal end of the tubular member. The first anchor may include a footplate. The first anchor may include a nitinol braid and/or polymer configured to flare into a deployed configuration. The first anchor may include an inflatable balloon. The inflatable balloon may be operably coupled to a fluid source. The first anchor may be configured for atraumatic apposition to the inner wall of the vessel. When the first anchor is in a deployed configuration, the first anchor may be configured to be oriented perpendicular to a central axis of the tubular member.

The tubular member may include a cannula or sheath.

The device may include a second tubular member disposed along the tubular member. The second tubular member may be operably coupled to the first anchor to move the first anchor from a delivery configuration to a deployed configuration.

The device may include a second anchor. The second anchor may be configured to placed on an outside surface of the vessel. A first anchor and a second anchor may cooperate to hold the tubular member to the vessel. The second anchor may include a suture pad. The second anchor may include an expandable tip. The second anchor may include nitinol braid or polymer.

The first and second anchors may pinch or sandwich the vessel to hold the tubular member to the vessel. The first and second anchors may include the same features. The first and second anchors may include different features.

In various aspects, a device may be provided. The device may include an anchor configured to hold a position of a tubular member to a vessel. The anchor may be positionable within the vessel. The anchor may include a proximal anchor portion configured to engage with subcutaneous tissue. The anchor may include a distal anchor portion configured to appose an inner wall of the vessel.

The distal anchor portion may include one or more fingers. The distal anchor portion may include a footplate. The distal anchor portion may include one or more barbs. The proximal anchor may include one or more barbs. The one or more barbs of the proximal anchor may be sharper than the one or more barbs of the distal anchor. Each of the distal and proximal anchors may include an atraumatic disk.

In various aspects, a method may be provided. The method may include inserting a tubular member into a vessel of a patient through an access site. The tubular member may extend at least partially into the vessel. The method may include holding a position of the tubular member to the vessel via a first anchor. The first anchor may be configured to appose an inner wall of the vessel.

The method may include inserting a medical device into the patient via the tubular member. The method may include, before the step of holding, inserting the first anchor into the vasculature in a delivery configuration. The method may include, before the step of holding, moving the first anchor to the deployed configuration in which a diameter of the first anchor in the deployed configuration may be greater than a diameter of the opening through which the tubular member is inserted into the vessel.

The method may include returning the first anchor to the delivery configuration and removing the tubular member and first anchor from the patient's vessel.

The step of holding may include holding a position of the tubular member to the vessel via a first anchor and a second anchor. The second anchor may be positioned on an outside of the vessel. The first and second anchors may cooperate with one another to hold the position of the tubular member to the vessel.

The step of holding may include pinching or sandwiching the vessel between the first and second anchors. The first anchor may include a distal anchor portion and a proximal anchor portion. The distal anchor portion may be configured to appose an internal wall of the vessel. The proximal anchor portion may be configured to engage with subcutaneous tissue.

### BRIEF DESCRIPTION OF FIGURES

FIG. 1 is a drawing of a sheath with arteriotomy locating feature and hole for blood.

FIG. 2 is a drawing of a malpositioned (proximal) hole.

FIG. 3 is a drawing of a malpositioned (distal) hole.

FIG. 4 is a drawing of a retracted sheath window.

FIG. 5 is a drawing of a device secured in an artery.

FIG. 6 is a drawing of a soft distal tip, showing its starting (solid lines) and ending (dotted lines) expansion.

FIG. 7 is a drawing of a device secured in an artery.

FIG. 8 is a drawing of a sheath with a footplate.

FIG. 9 is a drawing of features for securing a sheath.

FIG. 10 is a drawing of nitinol with covering features.

FIGS. 11A and 11B are a drawing showing the states of an expanding feature to locate an arteriotomy.

FIG. 12 is a drawing showing inner and outer shafts to change feature shape.

FIGS. 13A-13C are photographs showing braid/TPU located mid shaft un-expanded (13A), expanded (13B), and in proximity to hole (13C).

FIGS. 14A-14B are photographs showing braid/TPU at tip non-expanded (14A) and expanded (14B).

FIG. 15A shows embodiments having distal and proximal anchors.

FIGS. 15B-15D are profile views of embodiments of different expanding feature configurations.

FIG. 16A is an illustration of an embodiment of an expanding feature configuration.

FIGS. 16B-16P illustrate various examples of double barb designs (some in perspective view and others in cross-sectional planar views.

FIGS. 17A and 17B illustrate a double crush design.

FIGS. 18A and 18B are images showing additional embodiments of an anchor.

FIGS. 19A-19C are graphical depictions of an embodiment of a method for using devices with anchors.

### DETAILED DESCRIPTION

As is known, limb ischemia is a rapid and sudden decrease in a patient's limb perfusion, which may occur because of a blockage of blood due to an indwelling sheath and/or catheter, a local occlusion (e.g., an atherosclerotic narrowing), and/or a continuous occlusion resulting from small vessels and/or large sheaths. Limb ischemia also may occur as a result of a closure issue. To overcome such instances of limb ischemia, a clinician may need to allow for and/or create a path for flow past and/or around the sheath to deliver oxygenated blood to the distal limb of the patient.

Currently there are not many solutions on the market to address distal limb ischemia, such as when using larger diameter catheters or introducers in arteries. For example, introducer system today typically do not allow for placement of devices and removal from the vasculature without needing significant solutions to solve for bleeding at the arteriotomy site. Such solutions offer a way for the physician to place a larger bore device, such as an IMPELLA® heart pump, through the introducer and then pull the introducer back to allow for blood flow to get to the distal limb while still sealing at the arteriotomy.

Currently the physicians can pull traditional introducer systems back under fluoroscopy until just the tip is through the vessel wall, however, with no anchor in place. there is significant risk of the sheath tip pulling out of the vessel altogether leading to bleeding and possible death. Many patients with IMPELLA® heart pump or other devices may be sent from the Cath Lab to the ICU where they may be on support for long durations. In these settings, the introducers may not be monitored, and their position stability is important.

The inventors have appreciated the benefits of a sheath (e.g., an introducer sheath) that does not block a vessel or can be substantially removed from the vessel while still maintaining a connection, such as via a fixation feature (e.g., an anchor). In some embodiments, the sheath may be configured to allow for flow past and/or around the sheath to deliver oxygenated blood to the distal limb of the patient.

In view of the above, disclosed solutions may offer a means to allow a clinician (e.g., a physician) to locate a distal tip of the sheath relative to the vessel arteriotomy, such as by the anchor. The disclosed solutions also may offer a means to fixate the sheath to the vessel, such as via the anchor. As will be appreciated, the anchor may include a delivery configuration (e.g., for inserting the sheath into the vessel) and a deployed configuration in which the anchor fixates the sheath to the vessel. In some embodiments, the solution may allow the vessel to be fixated in place by pinching it against the artery wall from the inside and outside. In some embodiments, the anchor may include a nitinol braid, a balloon, and/or a footplate that may move between the delivery and deployed configuration.

In still other embodiments, the solutions described herein may offer a way for a clinician to remove the sheath at the end of the procedure without needing a full surgical cut down procedure. For example, the physician may first remove the catheter from the introducer shaft, after which the anchor may be returned to the delivery configuration, allowing the introducer shaft (and anchor from the patient). In on embodiment, the anchor may retract such that the sheath may return to a generally tubular shape. In an illustrative embodiment, the anchor may include a nitinol braid that may revert back down to a tubular shape, a balloon that may deflate back down to the tubular shape, and/or a footplate that may fold inward, allowing the physician to pulls the device from the anatomy.

In some embodiments, the anchor may be disposed at or near a distal tip of an introducer sheath through which a mechanical circulatory support device (“MCS”), such as a percutaneous blood pump, may be inserted. In other embodiments, the anchor may be included on an accessory sheath, such as a repositioning sheath, which may engage with the introducer sheath to fixate the introducer sheath and the repositioning sheath to the vasculature. In such embodiments, the repositioning sheath may be placed inside the introducer sheath. In such embodiments, the repositioning sheath may assist in occluding space between the MCS and the introducer sheath, which may prevent blood stagnation and clot formation in such locations.

Also disclosed herein are other manners to create a flow path around a sheath to deliver oxygenated blood to the distal limb of the patient. For example, in conventional systems, the sheath or other tubular member may be configured to have a small window to facilitate placement of the hole such that blood may flow from the tip of the sheath, through the sheath body, and out the hole to the distal artery. As will be appreciated in such an example, if the hole is aligned too far proximal outside the artery, the patient may bleed into subcutaneous tissue. If the hole is aligned too far distally, it will be against the vessel wall and blood will not be able to flow.

Difference in patient anatomy (vessel size, vessel depth) may make it difficult to design an all-in-one solution. The size of the window the hole must be in makes it so the slightest misplacement by the physician or migration of the sheath over time due to patient movement or site maintenance (cleaning, flushing, etc.) could cause one of the failure conditions above.

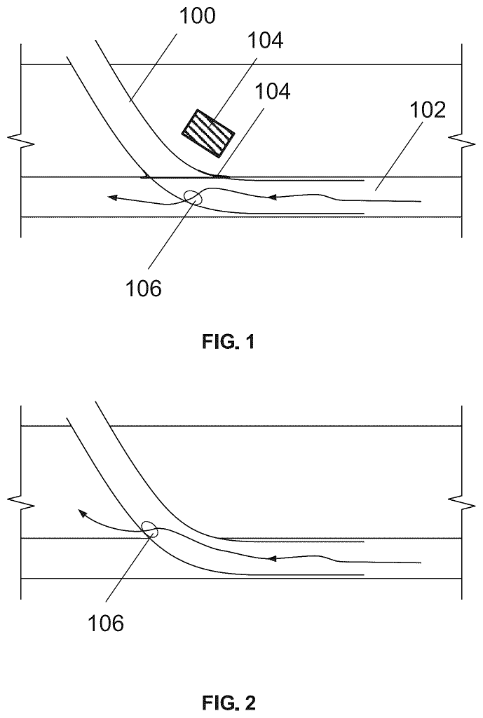

Turning now to the figures, FIG. 1-3 illustrate the challenges that may be experienced when trying to security and reliably locate a distal flow enabling feature in a very small “window” to avoid severe bleeding complications with varying patient anatomy exits. As shown in these views, the sheath 100 may be positioned in the patient's vasculature 102 (e.g., artery) with a feature 104 that is configured to locate the arteriotomy and prevent the sheath from coming out any further. Distal is a hole 106 that may allow blood to pass into the tip of the sheath coming from proximal artery, through the sheath, and out the hole past the sheath to distal artery. Window 105 illustrates the location of the hole 106 with respect to the arteriotomy that will allow for effective use. As will be appreciated in such instances, the sheath can completely occlude the vessel even when properly installed in the vasculature.

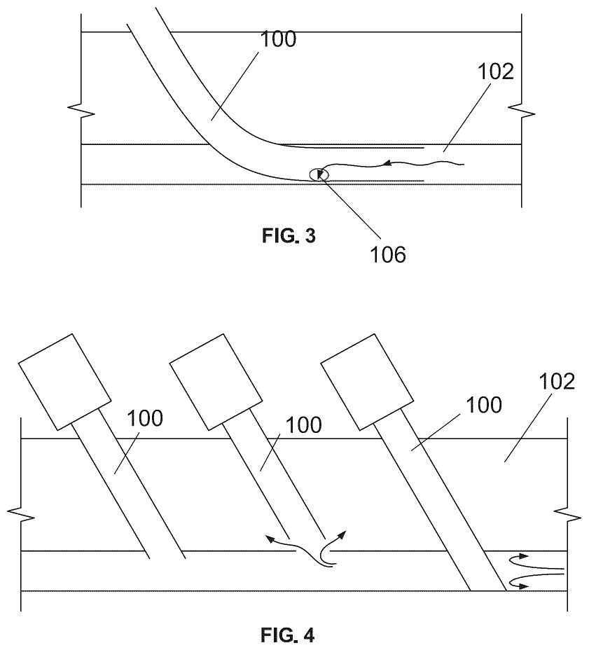

As seen in FIG. 2, with a malpositioned (proximal) hole 106, the hole slips out of the artery and blood can flows (see arrow) into subcutaneous tissue causing hematoma instead of flowing to distal artery. As seen in FIG. 3, with a malpositioned (distal) hole 106, the hole is advanced too far into the artery, rests against the vessel wall, and does not allow blood to flow (see arrow) into the sheath tip, through the sheath shaft, and out the hole causing acute limb (e.g., leg, arm, etc.) ischemia.

As appreciated by the inventors, trying to position the sheath tip just perfectly inside the vessel provides a small window for error. If the tip is too far proximal, blood will ooze out of the arteriotomy into the subcutaneous tissue. If it is too far forward, the sheath tip will be against the vessel wall blocking blood flow and potentially causing vascular injury.

As seen in FIG. 4, the sheath 100 may be positioned so access and hemostasis are maintained and blood flows past the sheath (see left sheath). If the sheath 100 is retracted too far, it can lose access and cause blood to flow out of the arteriotomy and into subcutaneous tissue causing a hematoma (see middle sheath). If the sheath 100 is pushed too far forward, it can block blood flow (e.g., by contacting the wall of the vasculature), which can cause acute limb ischemia (see right sheath).

In view of the above, the inventors have recognized the benefits of a device that can securely and reliably locate the sheath relative to an arteriotomy to promote distal limb perfusion. In some embodiments, the device may include an anchor, which may include one or more features, such as one or more mechanical features to locate the sheath relative to the arteriotomy and hold the position of the sheath relative to the arteriotomy. In some embodiments, the anchor may provide user feedback to allow a user to know the position of the sheath relative to the arteriotomy. In some embodiments, the anchor may include a set of mechanical features, with one of them being positioned in the lumen and a second being positioned outside the lumen. In such embodiments, the features may prevent acute limb ischemia by maintaining distal perfusion to the peripherals.

### First Mechanical Feature (Intra-Luminal)

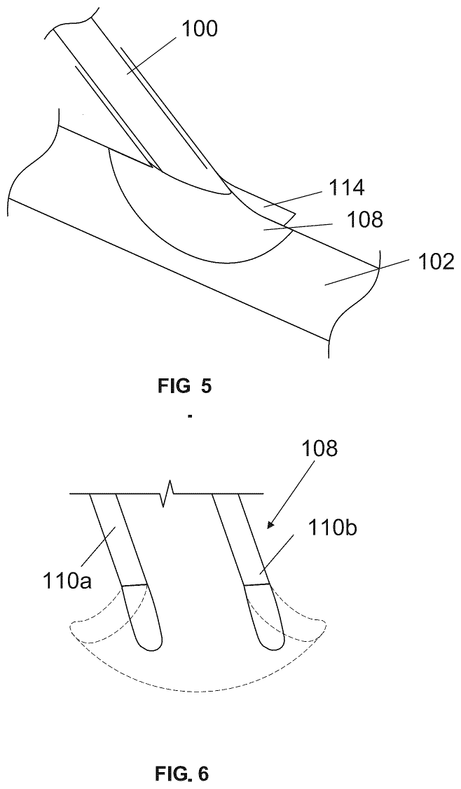

A first mechanical feature, anchor 108, may be on the sheath body 100 shaft (e.g., at a distal end) and may be configured to locate the arteriotomy by activating it and withdrawing the sheath against the inner wall of the artery (see, e.g., FIG. 5). As will be appreciated, in such embodiments, the anchor 108 may be configured to have a delivery configuration such that the anchor may pass through the arteriotomy, and a deployed configuration in which the anchor has a cross sectional area that exceeds the size of the arteriotomy such that withdrawing the sheath may place the anchor in apposition to the inner wall of the artery (locating the sheath in its desired location).

In some embodiments, like that shown in FIG. 5, the anchor may include a single mechanical feature. In other embodiments, as shown in FIG. 6, the anchor may include one or more fingers 110a, 110b, that may move between a delivery configuration (solid lines) and a deployed configuration (dashed lines). As will be appreciated, the fingers of the anchor may be moved into the deployed configuration once the sheath is in the vessel. In the deployed configuration, the fingers may be configured to appose and provide resistance against the inner wall of the vessel.

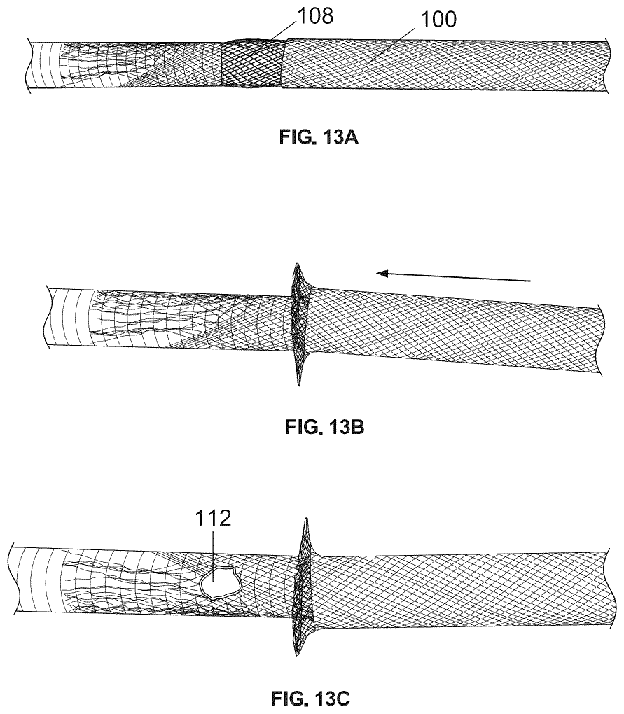

In some embodiments (see, e.g., FIGS. 13A-13C), the anchor 108 may include a nitinol braid and polymer that is a tube when free during insertion (e.g., see the delivery configuration of FIG. 13A) and is flared (see FIG. 13B) to the desired shape when axially compressed after delivered into the vessel (e.g., in the deployed position). In some embodiments, the nitinol braid and polymer that is set to the desired shape when in the deployed configuration (pre-set shape) and is held in tension or resides within shaft during insertion (e.g., in the delivery configuration). In that regard, in some embodiments, the anchor may be configured to automatically flare from the delivery position to the deployed position upon insertion into the vessel. In other embodiments, the clinician may need to selectively flare the anchor into the delivery position (e.g., by moving a first portion of the sheath relative to a second portion of the sheath, or by moving the sheath in a direction opposite to the direction of insertion).

As will be appreciated, the expanded region may have a diameter that exceeds that of the arteriotomy and that is configured to appose the inner wall of the vessel at the arteriotomy. Although not shown, it will be appreciated that when the sheath 102 of FIG. 13A-13C is properly located, the anchor is configured to appose the vessel wall at the anastomosis. As shown in FIG. 13C, the sheath may include an opening 112, distally located relative to the adjustable feature, to allow blood flow through the sheath.

In some embodiments, a laser-cut nitinol stent-like tube may be used in place the above-mentioned braids. As will be appreciated, the nitinol braid and/or laser-cut nitinol stent may have any suitable arrangement.

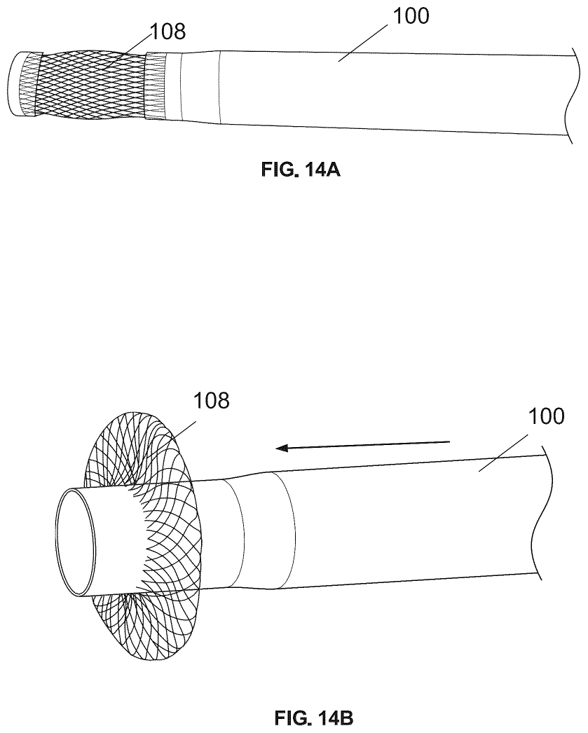

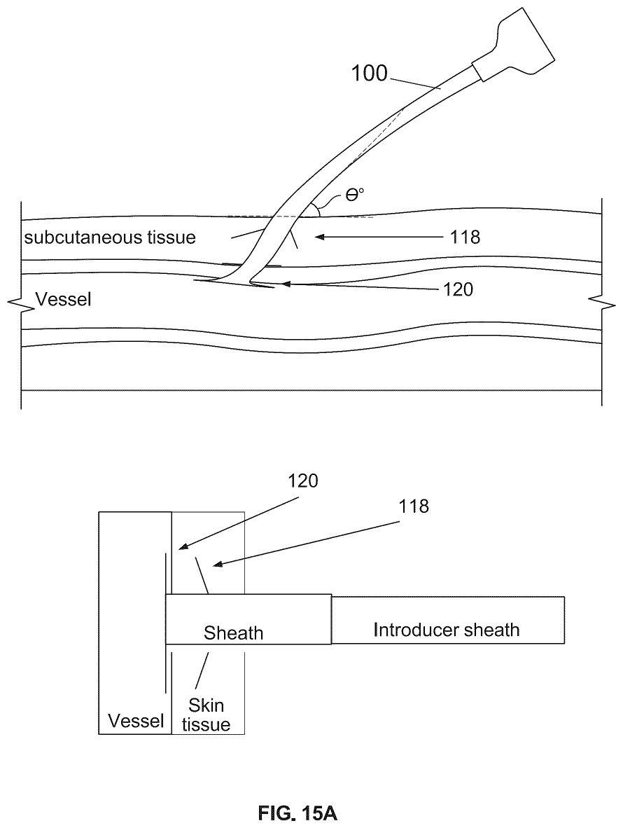

FIG. 14A and 14B illustrate another anchor example with an expandable nitinol region. In such embodiments, the expandable region (the deployed configuration of which is seen in FIG. 14B), is configured to be near the distal tip such that there is little to no sheath extending in the vasculature. In some embodiments, anchor (the adjustable feature) may include 72-end, 1×1, 75 μm wire (C26) w/ cilbond, and Carbothane PC-3575 0.080 mm laminated extrusion. The outer shaft used to force the shape change is composed of Pebax.

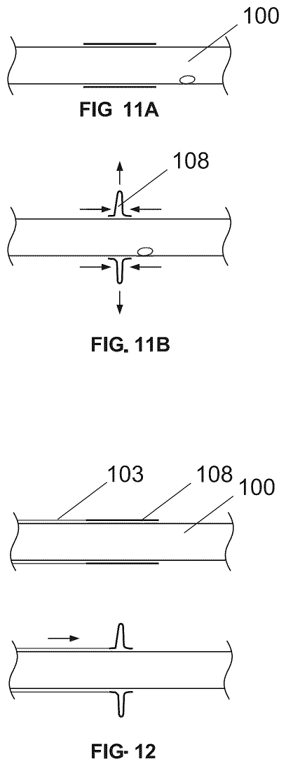

In FIGS. 11A and 11B, the states of an expanding feature to locate an arteriotomy can be seen. A sheath shaft 102 and mechanical feature of the anchor 108 in a delivery state (for insertion) are shown where it lays as a flat tube (FIG. 11A). A sheath shaft 102 and mechanical feature of the anchor 108 in a delivery state (for locating arteriotomy) are shown in FIG. 11B, where its length has been compressed and reduced and its diameter bulges and expands (bottom).

In FIG. 12, inner and outer shafts to change feature shape are seen. An inner sheath shaft 102 and outer sheath shaft 103 and an anchor 108 for locating arteriotomy may be included. The anchor may be fixed on its distal end (right) to the inner shaft 102. The anchor may be fixed on its proximal end (left) to the outer shaft 103 such that it can slide along the inner shaft. The assembly can be seen in its delivery position, in which the anchor lays flat. The outer shaft may then slide distally (see arrow), compressing the anchor and causing its length to reduce and its diameter to increase. As will be appreciated, the anchor can be positioned to appose the inner wall of a vessel. As will be further appreciated, the inner shaft may operate as an actuator to actuate expansion of the from the delivery configuration to the deployed configuration.

In some embodiments, the anchor (e.g., the adjustable feature) may be configured for atraumatic apposition to an inner surface of a blood vessel. In some embodiments, the anchor may be configured to conform to the blood vessel diameter.

In some embodiments, the anchor may be configured to be oriented perpendicular to the central axis of the inner sheath. In some embodiments, the anchor may be configured to be oriented at an angle relative to the central axis of the inner sheath that is 30-60 degrees. In some embodiments, the anchor may be configured to be oriented at an angle relative to the central axis of the inner sheath that is 45 degrees.

In some embodiments, the anchor may be configured to expand to have an outer diameter of 8-10 mm. In some embodiments, the anchor may be configured, when in a compressed state, to have a thickness of no more than 0.4 mm. In some embodiments, the anchor is fully reversible, so as to be compressible to a diameter smaller than the pump sheath inner diameter and configured to be removed through the pump sheath.

In some embodiments, the inner sheath may have a maximum outer diameter of no more than 5.05 mm. In some embodiments, the inner sheath may have an inner diameter of 2.5-3.5 mm, such as 3.0 mm.

In some embodiments, the first mechanical feature, anchor 108, may include a shaped balloon that flares to the desired shape when pressurized with fluid (e.g., to the delivery configuration). In such embodiments, the anchor may be fluidly coupled to a fluid source, and may be positioned at or near a tip of the sheath 100. As will be appreciated, as with the other embodiments disclosed herein, the balloon also may be configured to appose an inner wall of the vasculature and have a diameter that exceeds that of the access opening.

In still other embodiments, the anchor may include series of nitinol wires that again can be moved between a delivery position and a deployed configuration and again may be configured to appose an inner wall of the patient's vasculature.

In FIG. 8, a device is shown as being prevented from coming out of the artery by first anchor 108, which includes, e.g., a foot plate with a larger cross-section than the arteriotomy. As will be appreciated, such a foot play may include one or more mechanical features (e.g., fingers) that may move into a deployed configuration that may create the cross-section that is larger than the arteriotomy, once in the vessel, to hold the sheath relative to the arteriotomy. As will be appreciated, the footplate may have any suitable shape.

As will be appreciated, in view of the above, the first mechanical feature, anchor 108, may be located in various positions along the sheath. For example, the anchor may reside at the tip of the sheath so that the sheath lies substantially outside the vessel while maintaining hemostasis and access. As will be appreciated, in some embodiments, this may minimize the amount of sheath that may extend into the vasculature (e.g., possible causing an ischemic event), while still allow a medical device (e.g., a mechanical circulatory support device) to be passed into the patient. In other embodiments, the anchor may reside mid-shaft of the sheath body, in close proximity to a distally adjacent hole (see, e.g., hole 112 in FIG. 13C) that allows for blood to enter the sheath tip and pass through the hole to distal artery.

### Second Mechanical Feature (Extra-Luminal)

In some embodiments, the sheath body may include a second mechanical feature, or second lumen, which may be positioned extra-luminal. In some embodiments, the second anchor, may be configured to hold the sheath to the vessel. In some embodiments, the first and second anchors may work in concert to hold the sheath to the vessel at the arteriotomy. In this regard, the first and second anchors may have a press fit, snap fit, or other suitable engagement (e.g., openings and corresponding pins) to allow the first and second anchors to hold to one another. As will be appreciated, the anchor need not include such a second anchor, as described above, and may instead be locked at the arteriotomy via only the first anchor.

In some embodiments, the second mechanical feature may include a component such as ae.g. suture pad that may normally be locked in place and may slide on the sheath body shaft when actively activated (e.g., a button is pressed), and may returns to its normal locked in place configuration. In some embodiments, the component may be secured to a patient (e.g., via sutures).

The second anchor also may include a tube with a soft, expandable tip that when slid towards the first anchor and makes contact with the outer wall of the artery flares/expands open and opposes the soft tip to the outer wall of the vessel.

In still another embodiment, the second anchor may include a nitinol braid and polymer that is a tube when free during insertion and flares to the desired shape when axially compressed after delivered outside the vessel within subcutaneous tissue.

In some embodiments, the second anchor may include a nitinol braid and polymer that is set to the desired shape when free and is held in tension during insertion.

In still other some embodiments, a laser-cut nitinol stent-like tube may be used for the second mechanical feature. A shaped balloon may be used as the second mechanical feature, the balloon being capable of flaring to a desired shape when pressurized with fluid after delivered outside the vessel within subcutaneous tissue. As with the first mechanical feature, the second mechanical feature also may be configured to move between a delivery configuration and a deployed configuration.

As will be appreciated, the second anchor may be positioned in various locations. For example, in some embodiments, the second mechanical feature may be on the sheath body shaft and may have the purpose to secure sheath in place by providing a counter tension with the first mechanical feature. In some embodiments, the second mechanical feature also may be positioned proximally to the first and secures the sheath in place by “pinching” the vessel between the first and second mechanical feature.

In still other embodiments, the second mechanical feature may be positioned proximally to the first and secures the sheath in place by “pinching” the skin/sub-cutaneous tissue/vessel between the first and second mechanical feature.

As will be appreciated, in some embodiments, the first and second mechanical features may have the same features and/or arrangements (e.g., similar to those shown above with respect to the first mechanical feature), although the first and second mechanical features may differ. As will be further appreciated, in some embodiments, the first and second mechanical features also may include first and second anchors (with each including one or more mechanical features).

As seen in FIG. 5, a device such as the device from FIG. 3 may be shown inserted into the artery with an embodiment of a first mechanical feature 108 being rounded and conforming to the arterial diameter. It may be secured in place by the second mechanical 114 feature, which may include a tube with a soft expandable tip that conforms to the outer surface of the artery. In this regard, the device may be secured in place by “sandwiching” the artery between the first and second anchors.

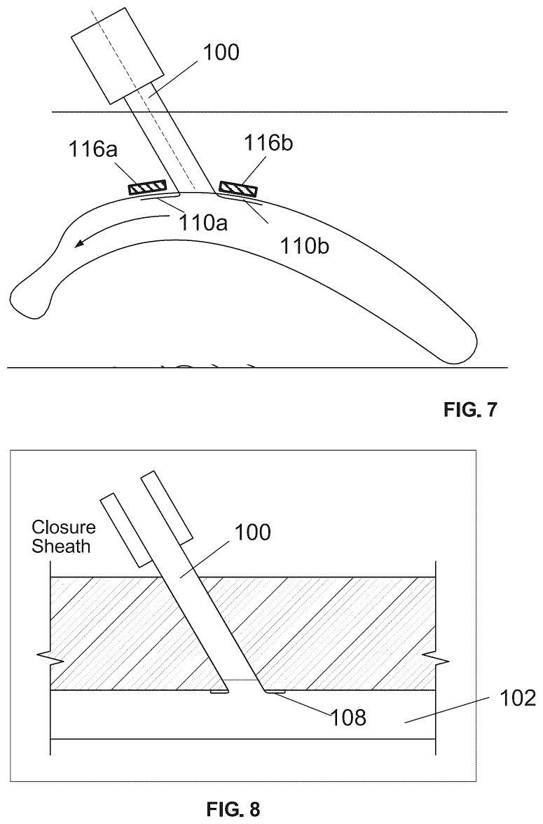

In FIG. 7, a device is seen secured by internal elements and external elements of the sheath (e.g., first and second anchors) that are pinching the artery to secure it in place. As shown in this view, the first and second anchors may have arrangements similar to those of the fingers shown in FIG. 6 (e.g., fingers 110a, 110b of the first anchor 108 and fingers 116a, 116b of the second anchor 114), with the inner and outer fingers engaging with each other to e.g. hold the sheath relative to the vessel.

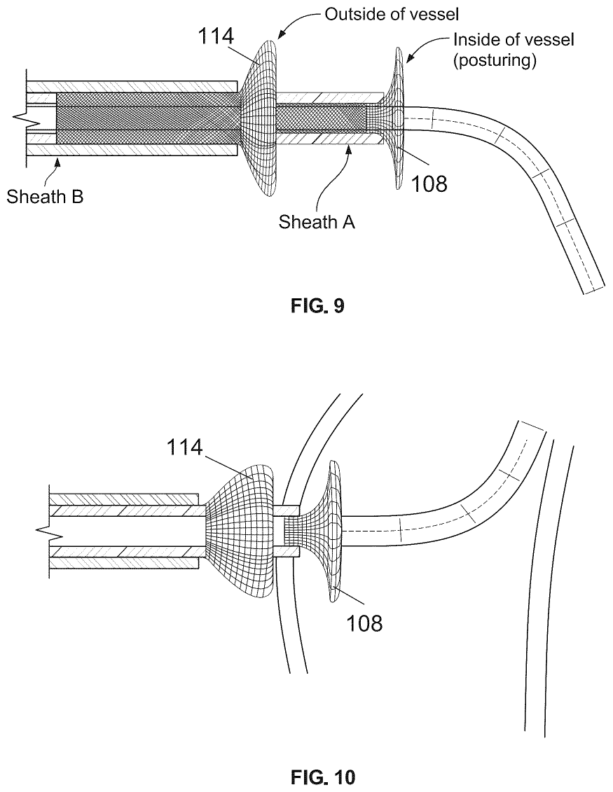

In FIG. 9, embodiments of an anchor for securing a sheath can be seen. In this embodiment, the first mechanical feature 108 with the internal sheath shaft (sheath A) tip and the second mechanical feature 114 with the external sheath shaft (sheath B) tip can be seen. When each sheath shaft is retracted proximally and exposes the mechanical elements, they may deform to a pre-set shape.

In FIG. 10, anchors having nitinol with covering features is shown. There, the first 108 and second 114 mechanical features are sheath sandwiching the vessel wall and may include a pre-set flared nitinol braid with a frame covering (e.g., ePTFE).

### Subcutaneous Anchor Design

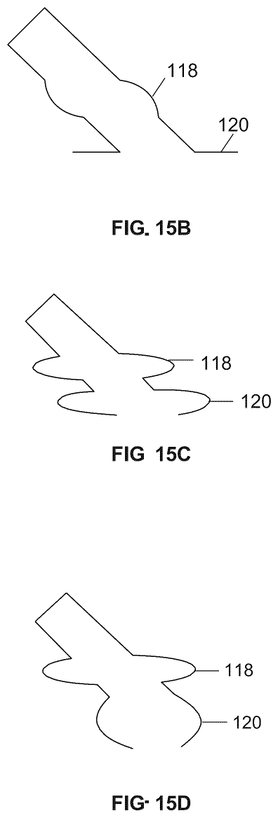

In some embodiments, as shown in FIG. 15A, the sheath 100 may be configured to include a proximal anchor 118 and a distal anchor 120. In such embodiments, the proximal anchor may be configured to engage with subcutaneous tissue while the distal anchor is configured to appose and provide resistance against the inner wall of the vessel. In this regard, the distal anchor 120 may include one or more of the configurations described above with respect to the first anchor 108. As will also be appreciated in view above, the proximal and distal anchors may also enable the anchor to hold the position of the sheath 100 relative to the vessel through. In some embodiments, the proximal and distal anchors may allow the sheath to maintain a desired angle θ of the sheath relative to the patient. In some embodiments, the angle may be between 30° and 60°, such an angle of 45°.

As will be appreciated in view of the figures show in FIG. 15B, 15C, and 15D, the proximal and distal anchors may have various different arrangements. As will be appreciated, on or both of the anchors may have designs similar to those described above with respect to the first and second anchors. For example, in 15B, the anchor may include a disk-barb design, where the distal anchor is configured to be atraumatic and have a shape set to confirm to the vessel. The proximal anchor is an atraumatic bump. In some embodiments, the atraumatic bump may include an inflatable balloon. The shape is retractable by passing another sheath over it, and the pull force for various embodiments may be ˜5N for distal barb retraction.

In FIG. 15B, a double-crush design can be seen. In such an embodiment, both the distal and proximal anchors may include both atraumatic discs.

In FIG. 15C, the anchors may include stent-disk design. For example, the distal anchor may have stent-like struts shaped to sit along a vessel's inner diameter. The proximal anchor may include an atraumatic disk (in other embodiments, it could be a bulge).

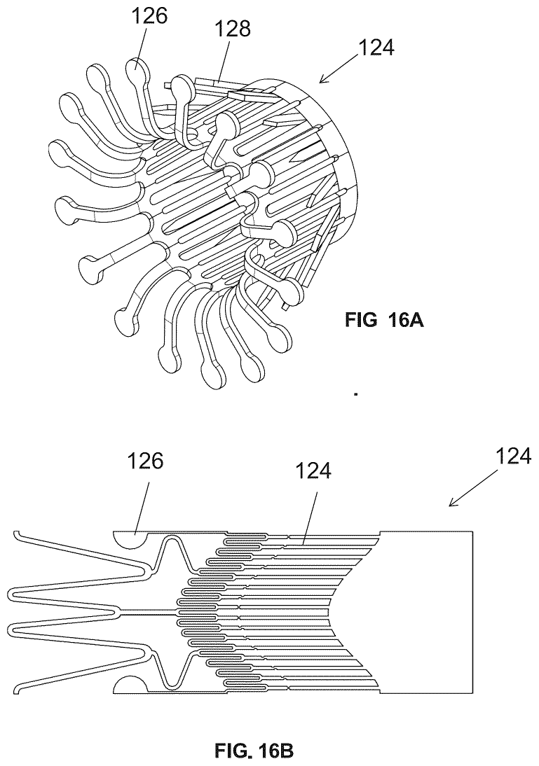

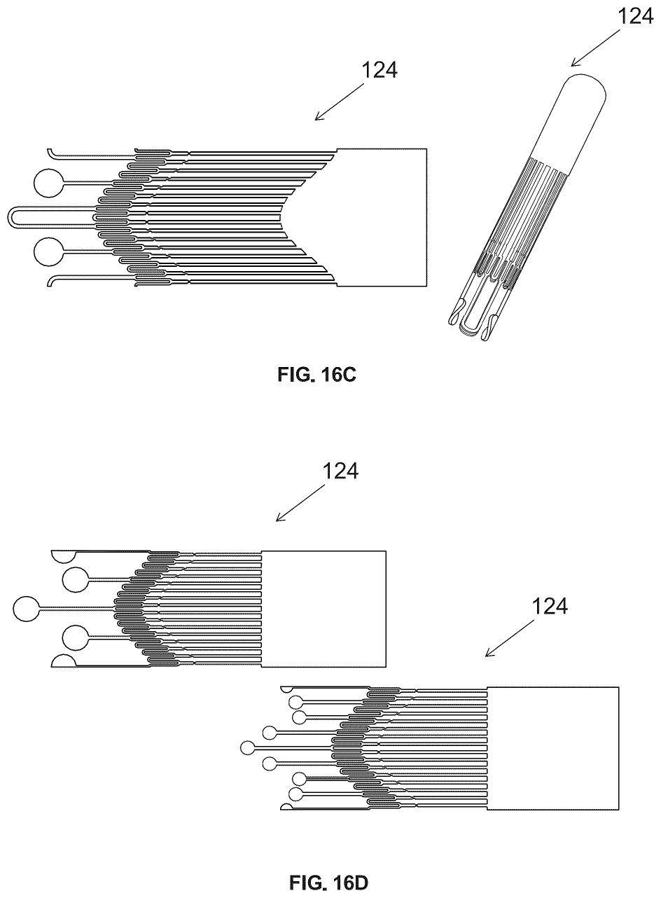

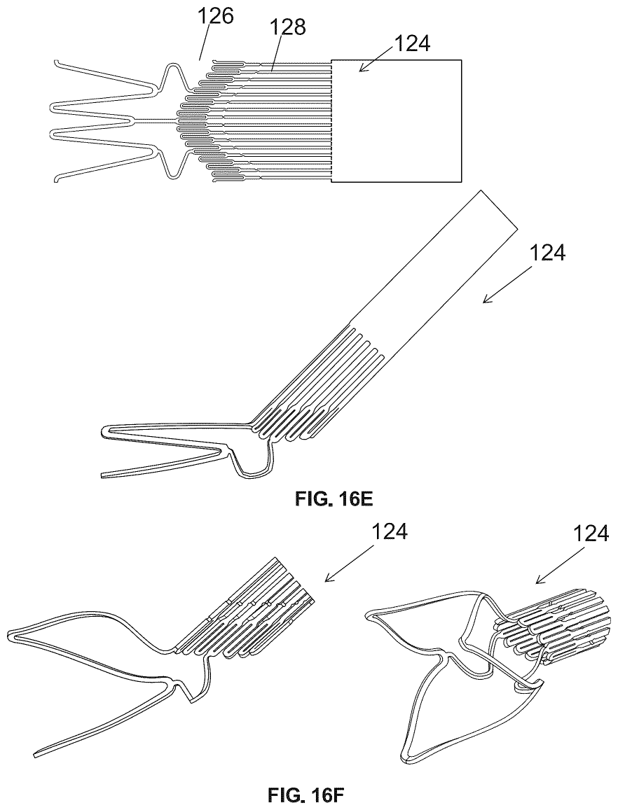

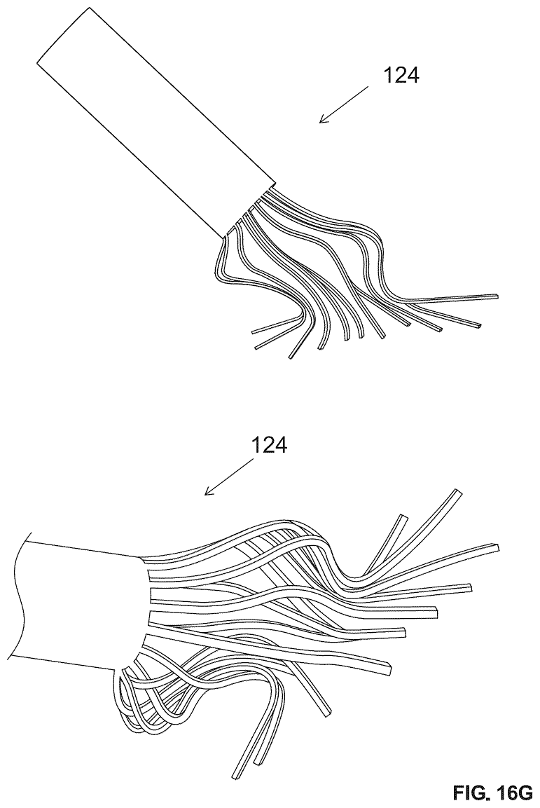

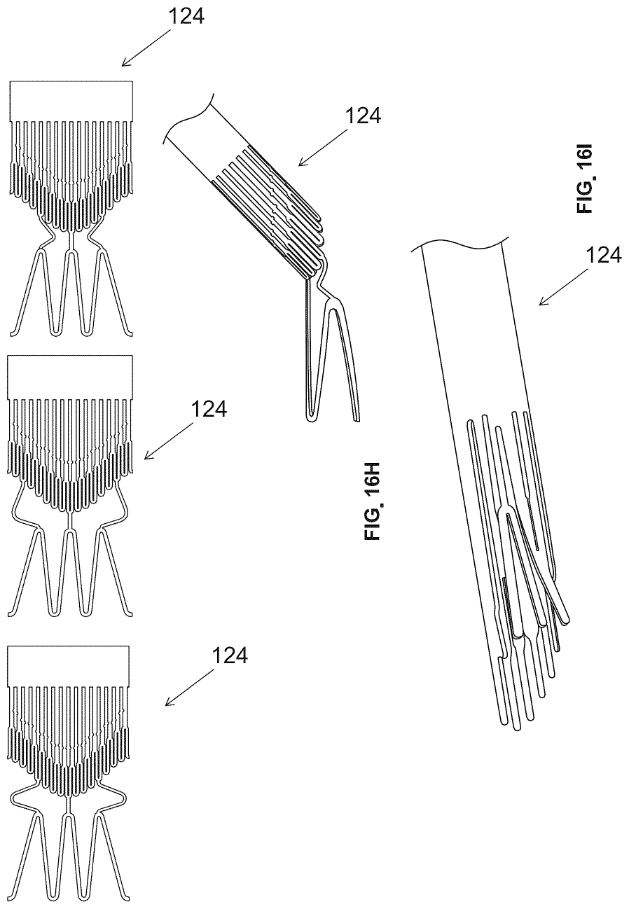

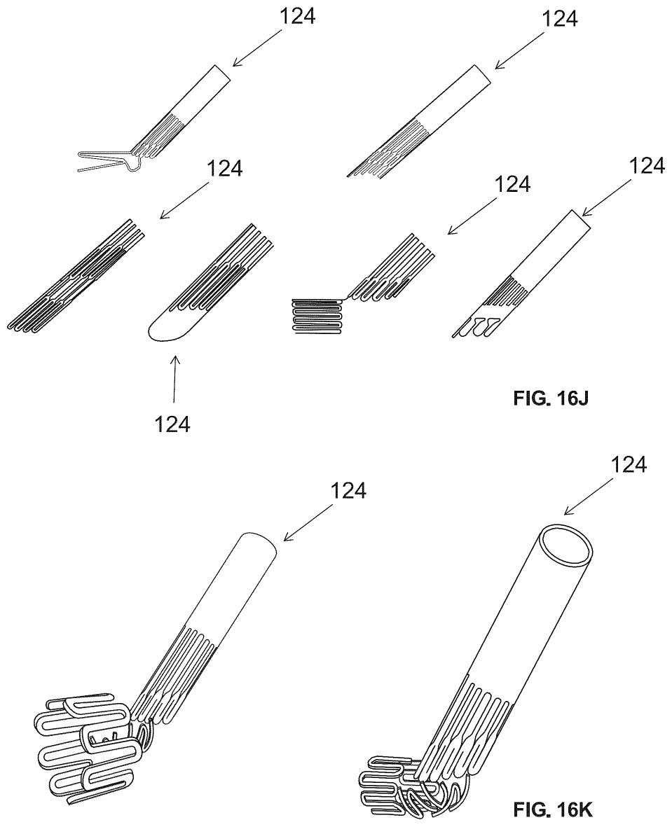

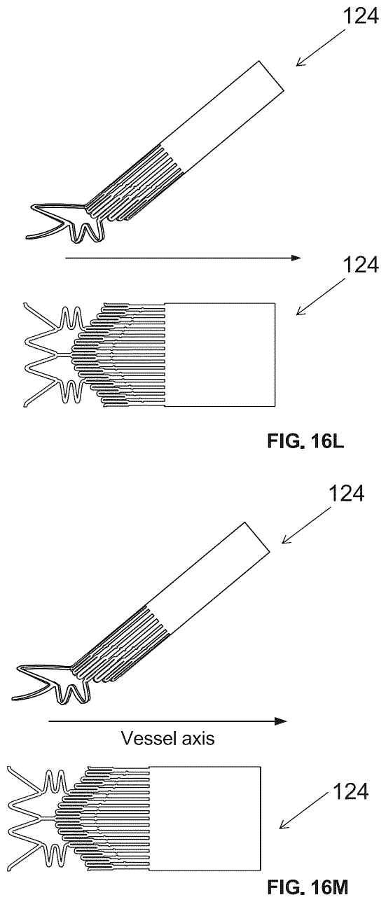

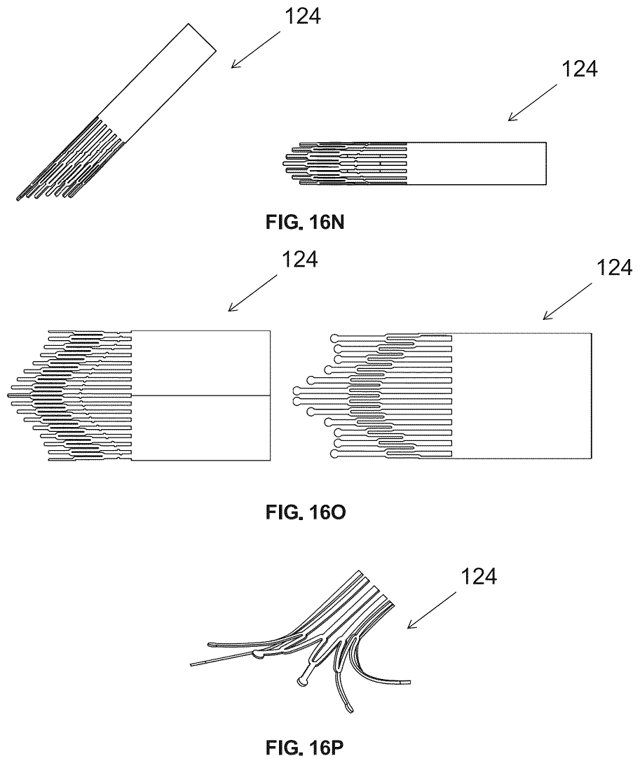

FIG. 16A-P illustrates various examples of anchors 124 having proximal and distal anchor portions 126, 128, also referred to herein as double anchors or double barb designs (some in perspective views and others in cross sectional planar views). In some embodiments, the distal anchor (e.g., the distal barbs) may be configured to be atraumatic and have a shape set to conform to the vessel. In some embodiments, the distal anchor may have a plurality of fingers that are moveable between a delivery and deployed configuration. In some embodiments, the fingers may include elongate members extending substantially parallel to a longitudinal axis of the anchor when in the delivery configuration. As will be appreciated, the fingers may all have the same configuration (e.g., length and shape), although configuration of the fingers may vary from finger to finger. In some embodiments, the fingers may splay outwardly and away from the longitudinal axis of the anchor. In some embodiments, the first and second anchors may be integrally formed with one another, although they may be joined in any suitable manner.

As with the above, in the deployed configuration, the distal anchor may appose the vessel wall. In contrast, the proximal barbs may be sharper, and may be configured as a engage (e.g., pierce) the, e.g., subcutaneous tissue. The distal and proximal anchor portions may have any suitable arrangement. In some embodiments, the anchor shown in these views may be used as either the first and/or second anchors disclosed herein.

As will be appreciated, the anchor may bet attached at different portions along the sheath, such as at or near a distal tip and/or at a portion of the sheath near an opening through which blood may flow.

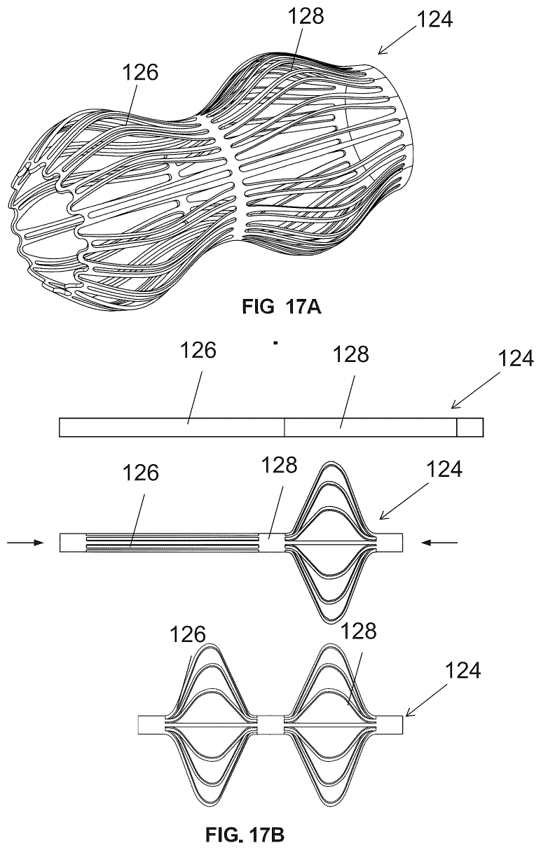

FIGS. 17A and 17B also illustrates a double crush design. For example, as shown in this view, the distal and proximal anchors both include atraumatic sections. As shown in FIGS. 17A and 17B, each of the anchors may be moveable between a compressed delivery position and an expanded deployed position.

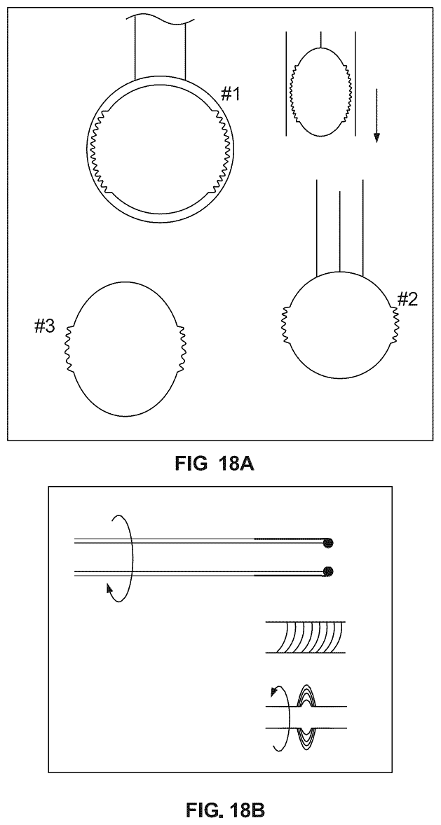

FIG. 18A also illustrates additional embodiments of anchors. As shown in these FIG. 18A, in some embodiments, the anchor may include a stent-like footplate which may disposed at a distal end of the sheath. In some embodiments, the anchor may not only be used to hold the sheath to the vessel (see #1 and #2, e.g., to allow for distal perfusion, the anchor also may remain in the vessel after the sheath is removed for closure (see #3). In this regard, the footplate may include a membrane. It also may include collogen doses, such as to facilitate closure.

FIG. 18A illustrates an example of rotational deployment of an anchor. As shown in this view, the anchor may have a coiled arrangement. It also may have one or more expandable portions.

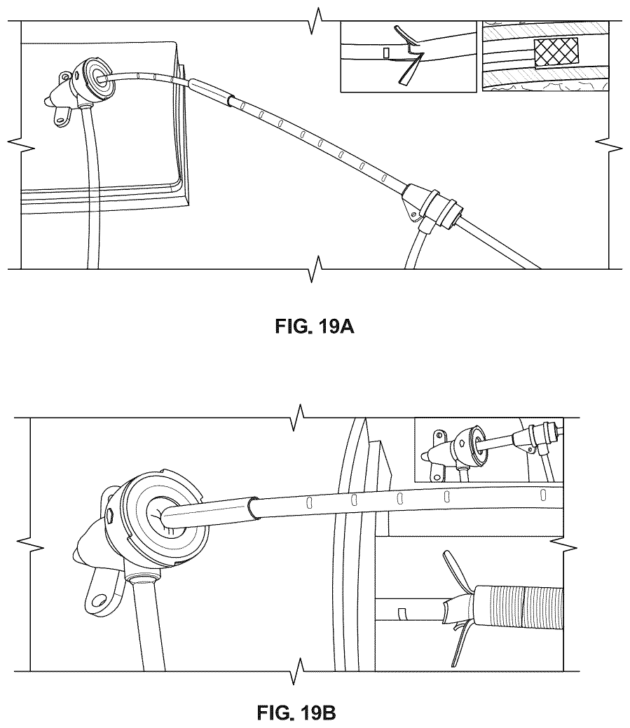

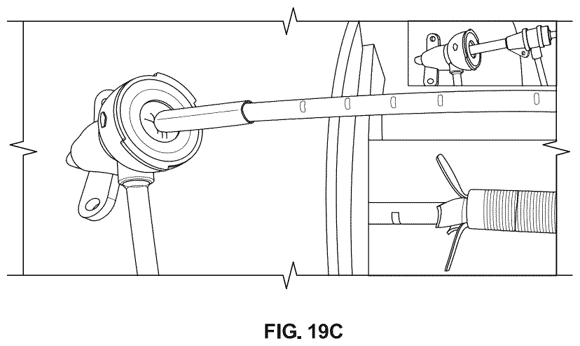

In an illustrated example (see FIGS. 19A-19C), to utilize some disclosed embodiments herein, a sheath may first be inserted over a dilator, and the dilator may be removed. In such embodiments, the sheath straightens and tortuous anatomy and/or bypasses calcification. Optionally, a medical device, such as a mechanical circulatory support device (e.g., a percutaneous pump), may be inserted and placed into an appropriate position (e.g., in a ventricle) via the sheath. Once the sheath (and optional medical device) is in place, the anchor may be deployed. If the sheath can be retracted, the retraction can occur at this time. The anchor may be apposed to the blood vessel. A second anchor may then be deployed to sandwich the vessel and/or subcutaneous barbs may be deployed to lock the anchor in place. In some embodiments, a Touhy butterfly may then be advanced to secure the anchor with compression from the skin. In some embodiments, in the case the sheath is a distal perfusion sheath, the sheath may be oriented perpendicular to a centerline of the blood vessel or normal to the surface of the skin. In some embodiments, the sheath may be oriented ±10 degrees from perpendicular to a centerline of the blood vessel or normal to the surface of the skin.

In another illustrated example, the device may include an introducer sheath. The device may be configured to have a first state, which may be used for, e.g., insertion of sheath with corresponding dilator and used to deliver a pump (e.g., an Impella® blood pump) through calcification, tortuosity, or other patient anatomy that could not otherwise pass the pump.

The first Mechanical feature may be a Nitinol braid, located concentrically around and at the distal tip of the inner sheath body. At the distal most portion may be fixed to the inner sheath body. The proximal end of the mechanical feature may be fixed to an outer sheath shaft that may slide along the inner sheath shaft. The Inner sheath shaft may have a liner (e.g., a PTFE liner), that may be coil reinforced, and may have an outer jacket. It may be attached on its proximal end to a sheath hub comprising a hemostatic valve and side-arm for flushing. The Outer sheath shaft may be attached on its proximal end to a molded hub that may be locked in two positions. In the first state, it may be located in a proximal position adjacent distally to the sheath hub of the inner shaft.

The second mechanical feature may be a catheter lock including a housing, button with spring, locking tube, and suture pad. It may be located concentrically on the outer shaft between its proximal end and distal end. In the first state, the button is not depressed so the spring creates a compressive force on the locking tube, fixing the second mechanical feature to the outer sheath shaft. The second mechanical feature may be in a proximal position adjacent distally to the molded hub of the outer sheath shaft. The introducer sheath may take up significant or complete luminal cross-sectional area with little to no flow getting past the sheath.

The device may be configured to have a second state, which may be used to, e.g., place the pump and initiate therapy. The sheath may be retracted partially out of the artery with the distal tip still within the arterial lumen. The first mechanical feature may be activated, and the sheath may be pulled in tension, so the mechanical feature opposes to the vessel wall without pulling out. The second mechanical feature may be activated, slide down to press with a force against the skin, and un-activated to fix in place on the shaft to provide a counter force to the first mechanical feature to secure the sheath in place. The molded hub of the outer shaft may be moved in a forward direction to a second position where it locks in place. The length of the first feature may reduce and the diameter may increase. The introducer sheath takes up limited luminal cross-sectional area with sufficient distal perfusion pas the sheath.

The device may be configured to have a third state, which may be used, e.g., when therapy is complete, and the user removes the pump from the sheath. A wire to maintain access may be inserted into the sheath. The first mechanical feature may be returned to an un-expanded state and the sheath may be removed. The arteriotomy is closed with common technique (manual pressure, vascular closure device, etc.). The hub of the outer shaft may be moved in a distal direction to the first position where it locks in place. The length of the first feature may increase back to its original length and the diameter substantially returns to its original diameter.

Although embodiments are shown and described in which the anchor(s) and or first and second mechanical features may be include on the introducer (e.g., introducer sheath for inserting the medical device), in some embodiments, the anchor may instead be formed on another accessory, such as a repositioning sheath, which is configured to engage with the introducer sheath.

In some embodiments, as shown in FIG. 19A in pre-deployment stage, the self-expandable pre-shaped anchor may be positioned at the tip of an inner sheath (e.g., a repositioning sheath). The inner sheath may include an anchor protection sleeve place around the anchor and optionally a portion of the inner sheath. As will be appreciated, the anchor protector sleeve may not be slideable into a hub and into the introducer (e.g., pump) sheath.

During deployment, as shown in FIG. 19B, repositioning sheath may be pushed into a pump sheath. The anchor protect sleeve will bridge the anchor through the hemostasis valve of the pump sheath, but the anchor protect sleeve will not enter into the pump sheath body. The anchor will self-deploy once it is pushed through the pump sheath tip. The repositioning sheath can then be pulled back (in a proximal direction) slightly, to make sure the anchor is taut against the pump sheath tip. The repositioning sheath position may then be locked by locking the feature at the pump sheath hub once the anchor is deployed. The pump sheath may then be retracted until resistance is felt, which indicates the anchor is apposed to the inner wall of the vessel. In some embodiments, an adjustable butterfly may be pushed forward to be pressed against the skin, then sutured in place, to hold the pump sheath in place.

The anchor can be undeployed by retracting the repositioning sheath.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

1. A device comprising:
a tubular member configured to extend at least partially into a vessel of a patient; and
a first anchor coupled to the tubular member and configured to hold a position of the tubular member relative to the vessel, wherein the first anchor is configured to appose an inner wall of the vessel.

2. The device of claim 1, wherein the first anchor includes a delivery configuration and a deployed configuration, wherein, in the deployed configuration, the first anchor has a diameter greater than an opening of the vessel through which the tubular member extends into the vessel.

3. The device of claim 1, wherein the first anchor includes a plurality of fingers.

4. The device of claim 1, wherein the first anchor is attached at or near a distal end of the tubular member.

5. The device of claim 1, wherein the first anchor includes a footplate.

6. The device of claim 1, wherein the first anchor includes a nitinol braid and/or polymer configured to flare into a deployed configuration.

7. The device of claim 1, wherein the first anchor includes an inflatable balloon.

8. The device of claim 7, wherein the inflatable balloon is operably coupled to a fluid source.

9. The device of claim 1, further comprising a second tubular member disposed along the tubular member, wherein the second tubular member is operably coupled to the first anchor to move the first anchor from a delivery configuration to a deployed configuration.

10. The device of claim 1, wherein the first anchor is configured for atraumatic apposition to the inner wall of the vessel.

11. The device of claim 1, wherein, when the first anchor is in a deployed configuration, the first anchor is configured to be oriented perpendicular to a central axis of the tubular member.

12. The device of claim 1, wherein the tubular member includes a cannula or sheath.

13. The device of claim 1, further comprising a second anchor configured to placed on an outside surface of the vessel, wherein a first anchor and a second anchor cooperate to hold the tubular member to the vessel.

14. The device of claim 13, wherein the second anchor includes a suture pad.

15. The device of claim 13, wherein the second anchor includes an expandable tip.

16. The device of claim 13, wherein the second anchor includes nitinol braid or polymer.

17. The device of claim 13, wherein the first and second anchors pinch or sandwich the vessel to hold the tubular member to the vessel.

18. The device of claim 13, wherein the first and second anchors include the same features.

19. The device of claim 13, wherein the first and second anchors include different features.

20. A device comprising:
an anchor configured to hold a position of a tubular member to a vessel, the anchor being positionable within the vessel, wherein the anchor includes:
a proximal anchor portion configured to engage with subcutaneous tissue; and
a distal anchor portion configured to appose an inner wall of the vessel.

21-22. (canceled)

23. The device of claim 20, wherein the distal anchor includes one or more barbs.

24. (canceled)

25. The device of claim 20, wherein each of the distal and proximal anchors include an atraumatic disk.

26. A method comprising:
inserting a tubular member into a vessel of a patient through an access site, the tubular member extending at least partially into the vessel; and
holding a position of the tubular member to the vessel via a first anchor, the first anchor configured to appose an inner wall of the vessel.

27-34. (canceled)
